(19) United States
(12) Patent Application Publication
(10) Pub. No.: US 2009/0143387 A1
(43) Pub. Date: Jun. 4, 2009

(54) SUSTAINED-RELEASE TABLET COMPOSITION

(75) Inventors: Gregory E. Amidon, Portage, MI (US); Loksidh D. Ganorkar, Kalamazoo, MI (US); John M. Heimlich, Portage, MI (US); Ernest J. Lee, Kalamazoo, MI (US); Alice C. Martino, Kalamazoo, MI (US); Robert M. Noack, Grand Rapids, MI (US); Joseph P. Reo, Kalamazoo, MI (US); Connie J. Skoug, Portage, MI (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 12/339,212

(22) Filed: Dec. 19, 2008

Related U.S. Application Data
(63) Continuation of application No. 10/626,379, filed on Jul. 24, 2003.
(60) Provisional application No. 60/398,427, filed on Jul. 25, 2002.
(60) Provisional application No. 60/398,447, filed on Jul. 25, 2002.
(60) Provisional application No. 60/406,609, filed on Aug. 28, 2002.
(60) Provisional application No. 60/479,387, filed on Jun. 18, 2003.

(51) Int. Cl. A61K 31/535; A61P 25/24

(52) U.S. Cl. 514/239.2

(57) ABSTRACT

A sustained-release pharmaceutical composition in a form of an orally deliverable tablet containing reboxetine, or a pharmaceutically acceptable salt thereof, dispersed in a matrix containing a hydrophilic polymer and a starch, wherein the starch has a tensile strength of at least 0.15 kN cm⁻² at a solid fraction of 0.75 to 0.85.

This application claims priority of U.S. provisional application Ser. No. 60/398,427 filed on Jul. 25, 2002; U.S. provisional application Ser. No. 60/398,447 filed on Jul. 25, 2002; U.S. provisional application Ser. No. 60/406,609 filed on Aug. 28, 2002; and U.S. provisional application Ser. No. 60/479,387 filed on Jun. 18, 2003.

### FIELD OF THE INVENTION

The present invention relates to tablet formulations, and more particularly to a sustained-release tablet composition for oral delivery of a water-soluble drug or prodrug.

### BACKGROUND OF THE INVENTION

Many active pharmaceutical agents, including drugs and prodrugs, have been formulated as orally deliverable dosage forms providing sustained release (otherwise known as slow release or extended release) of such agents over a period of time effective to permit once daily administration. A well-known system for formulating such dosage forms involves a matrix comprising a hydrophilic polymer wherein the agent is dispersed; the agent is released over a period of time in the gastrointestinal tract upon dissolution or erosion of the matrix. Sustained-release dosage forms comprising such a matrix system are conveniently prepared as compressed tablets, described herein as “matrix tablets”.

Drugs and prodrugs having relatively high solubility in water, for example a solubility of about 10 mg/ml or greater, present challenges to the formulator wishing to provide a sustained-release dosage form, and the higher the solubility the greater are the challenges. These challenges are well illustrated in the cases of pramipexole dihydrochloride, which has a solubility in water of about 200 mg/ml, and reboxetine mesylate, which has a solubility in water of about 250 mg/ml.

Pramipexole (I) is a dopamine D₂ receptor agonist useful in treatment of Parkinson's disease. Pramipexole as its dihydrochloride salt is commercially available in the United States as Mirapex® tablets of Pharmacia & Upjohn. These are immediate-release tablets in 0.125 mg, 0.25 mg, 0.5 mg, 1.0 mg and 1.5 mg strengths, designed for oral administration of a single tablet three times per day to provide a daily dose of 0.375 to 4.5 mg. See Physicians' Desk Reference 57th edition (2003), 2768-2772. Doses herein are expressed in amounts of pramipexole dihydrochloride monohydrate unless otherwise specified; 1.0 mg pramipexole dihydrochloride monohydrate is equivalent to about 0.7 mg pramipexole base.

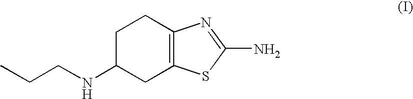

A three times daily dosing regimen for immediate-release pramipexole dihydrochloride tablets is well tolerated, but patient compliance would be much improved if a once-daily regimen were possible. In this regard, it will be noted that the primary indication for the drug, Parkinson's disease, is an affliction that becomes more prevalent with advancing age and is often accompanied by decline in memory. A once-daily regimen would be especially useful in enhancing compliance among elderly patients.

It has been found by the present inventors that formulation of pramipexole dihydrochloride monohydrate in a hydrophilic matrix tablet is generally inadequate to provide sustained-release properties consistent with once-daily dosing. Release characteristics can be further modified by coating the tablet with a sustained-release coating. Such a coating typically comprises a hydrophobic polymer and a hydrophilic pore-former.

The need to provide a coating over the matrix tablet gives rise to further problems. The additional handling operations involved in a coating step require a sufficient degree of tablet hardness to avoid tablet breakage and/or attrition during these operations, particularly in a high-speed manufacturing situation.

Reboxetine (II) is a selective noradrenaline reuptake inhibitor (SNRI) useful in treatment of depressive illness. Reboxetine as its methanesulfonate (mesylate) salt is commercially available in the United Kingdom and elsewhere as Edronax® tablets of Pharmacia & Upjohn. These are immediate-release tablets having a breaking score to facilitate division. Each Edronax® tablet contains 4 mg reboxetine and is designed for twice daily oral administration to provide a daily dose of 4 to 12 mg, with division of tablets if necessary. See British National Formulary 41st edition (2001), 196. Doses herein are expressed in amounts of reboxetine base unless otherwise specified.

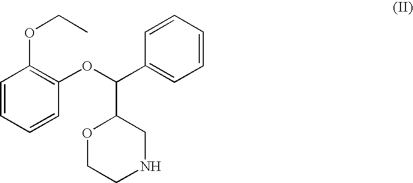

A twice-daily dosing regimen for immediate-release reboxetine tablets is well tolerated, but patient compliance would be much improved if a once-daily regimen were possible without substantially increasing the potential for adverse side effects. In this regard, it will be noted that the primary indication for the drug, depressive illness, is an affliction that is often accompanied by poor compliance.

It has been found by the present inventors that formulation of reboxetine salts in a hydrophilic matrix tablet can provide sustained-release properties consistent with once-daily dosing. However, the resulting tablets are susceptible to breakage and/or attrition during handling, especially in a high-speed tableting operation.

It has proved difficult to formulate a tablet having a suitable combination of sustained-release and handling properties, where the drug is one having relatively high solubility, as in the case of salts of pramipexole or reboxetine.

U.S. Pat. No. 6,197,339 discloses a sustained-release tablet comprising (R)-5,6-dihydro-5-(methylamino)-4H-imidazo[4,5-ij]-quinolin-2(1H)-one (Z)-2-butenedioate (1:1) (sumanirole maleate) in a matrix comprising hydroxypropylmethylcellulose (HPMC) and starch. The tablet is disclosed to be useful in treatment of Parkinson's disease. Starches disclosed to be suitable therein include pregelatinized starch.

U.S. Pat. No. 5,458,887 discloses a controlled-release tablet comprising an osmotic core that consists of a drug in admixture with a water-swellable component such as HPMC or polyethylene oxide, and a coating that comprises a water-resistant polymer and a minor amount of a water-soluble compound that acts as a pore-former. Upon formation of pores in the coating by dissolution of the water-soluble compound, the water-swellable agent is said to expand the core and provide a drug-rich surface in contact with gastrointestinal fluid.

U.S. Pat. No. 5,656,296 discloses a dual control sustained-release formulation comprising a core that comprises a drug and a low melting point excipient, and a coating layer over the core that comprises a pH-independent water-insoluble polymer and a water-soluble film-forming polymer.

European Patent Application No. EP 0 933 079 discloses a starch said to be suitable for preparing tablets having high hardness yet being capable of rapid disintegration in an aqueous medium. Tensile strength of the finished tablets is calculated from the hardness.

Patents and publications cited above are incorporated herein by reference.

It is an object of the present invention to provide a sustained-release tablet composition of a water-soluble drug or prodrug that is suitable for once-daily oral administration. It is a further object to provide such a composition having sufficient hardness to withstand a high-speed tableting operation, in particular to resist erosion during application of a coating layer. It is a further object to provide a pharmaceutical tablet that provides day-long therapeutic effect on the central nervous system (CNS) of a subject when administered once daily. It is particular object to provide such a tablet that provides day-long therapeutic effect as a dopamine agonist when administered once daily, most particularly where the water-soluble drug is a salt of pramipexole, without substantially increased incidence of adverse side effects. It is another particular object to provide such a tablet that provides therapeutic effect as an SNRI when administered once daily, most particularly where the water-soluble drug is a salt of reboxetine or an enantiomer thereof, for example (S,S)-reboxetine, without substantially increased incidence of adverse side effects. It is a still further object to provide a method for testing a starch to assess its suitability for inclusion in a matrix tablet for sustained release of a water-soluble drug or prodrug.

### SUMMARY OF THE INVENTION

There is now provided a sustained-release pharmaceutical composition in a form of an orally deliverable tablet comprising an active pharmaceutical agent having solubility not less than about 10 mg/ml, dispersed in a matrix comprising a hydrophilic polymer and a starch having a tensile strength of at least about 0.15 kN cm⁻² at a solid fraction representative of the tablet. The composition preferably exhibits sustained-release properties adequate to provide therapeutic effectiveness when administered orally not more than once daily to a subject in need thereof.

There is further provided a process for preparing a sustained-release pharmaceutical composition in a form of an orally deliverable tablet, the process comprising selecting by a suitable test a starch having a tensile strength of at least about 0.15 kN cm⁻² at a solid fraction representative of the tablet; admixing with the selected starch a hydrophilic polymer and an active pharmaceutical agent having solubility not less than about 10 mg/ml to provide a mixture wherein the agent is dispersed in a matrix comprising the polymer and the starch; and compressing the mixture to form a tablet.

A particularly convenient test method, which is itself a further embodiment of the invention, comprises preparing compacts of a starch sample on an automated tablet press at a range of compression forces, measuring hardness of the compacts, determining solid fraction of the compacts, calculating tensile strength of the compacts from hardness and dimensions of the compacts, determining relationship of tensile strength to solid fraction of the compacts, and from that relationship estimating tensile strength at a solid fraction representative of a desired tablet.

There is still further provided a method of treatment of a subject having a condition or disorder for which an active pharmaceutical agent having solubility not less than about 10 mg/ml is indicated, the method comprising orally administering to the subject a sustained-release pharmaceutical composition in a form of a tablet comprising the agent dispersed in a matrix comprising a hydrophilic polymer and a starch having a tensile strength of at least about 0.15 kN cm⁻² at a solid fraction representative of the tablet.

An “active pharmaceutical agent” herein can be a drug or a prodrug or a salt thereof, including diagnostic agents. Unless otherwise specified, “solubility” herein means solubility in water at 20-25° C. at any physiologically acceptable pH, for example at any pH in the range of about 4 to about 8. In the case of an agent that is a salt, reference herein to solubility in water pertains to the salt, not to the free acid or base form of the agent.

The term “orally deliverable” herein means suitable for oral, including peroral and intra-oral (e.g., sublingual or buccal) administration, but tablets of the present invention are adapted primarily for peroral administration, i.e., for swallowing, typically whole or broken, with the aid of water or other drinkable fluid.

A “compact” herein is a compressed tablet, prepared for example on a tablet press, consisting only of a sample of starch for which it is desired to measure tensile strength. “Solid fraction” is the ratio of absolute to apparent density of a compact. A “solid fraction representative of the tablet” is a solid fraction selected to be similar to the solid fraction of tablets prepared according to the invention. Typically a solid fraction of about 0.75 to about 0.85, illustratively 0.8, will be selected.

A “subject” herein is an animal of any species, preferably mammalian, most preferably human. Conditions and disorders in a subject for which a particular agent is said herein to be “indicated” are not restricted to conditions and disorders for which the agent has been expressly approved by a regulatory authority, but also include other conditions and disorders known or believed by a physician to be amenable to treatment with the agent. “Treatment” herein embraces prophylactic treatment unless the context requires otherwise.

### BRIEF DESCRIPTION OF THE DRAWINGS

FIG. 1 is a graph showing relationship of tensile strength of pregelatinized starch lots, as determined by a test method of the invention using a 4 second dwell time (Example 1 herein) to triaxial tensile strength.

FIG. 2 is a graph showing relationship of tensile strength of pregelatinized starch lots, as determined by a test method of the invention using a 90 second dwell time (Example 1 herein) to triaxial tensile strength.

FIG. 3 is a graph showing correlation of tensile strength of pregelatinized starch lots with maximum hardness of tablets containing these lots.

FIG. 4 is a graph showing in vitro dissolution profiles of three different 0.375 mg sustained-release tablet formulations of pramipexole dihydrochloride monohydrate, as more fully described in Example 10.

FIG. 5 is a graph showing in vitro dissolution profiles of three different 4 mg sustained-release tablet formulations of (S,S)-reboxetine in the form of its succinate salt, as more fully described in Example 12.

### DETAILED DESCRIPTION OF THE INVENTION

The invention provides a pharmaceutical composition in a form of an orally deliverable tablet comprising a water-soluble active pharmaceutical agent. The composition preferably exhibits sustained-release properties adequate to provide therapeutic effectiveness when administered orally not more than once daily.

Typically, agents for which the invention is especially useful are unsuitable for once daily administration when formulated in an immediate-release composition. This unsuitability can arise from one or more properties of such agents including without limitation:
(a) short half-life (T_{1/2}) of the agent or active metabolite thereof in the bloodstream, requiring the plasma concentration to be “topped up” at intervals shorter than one day to maintain a therapeutically effective concentration; and
(b) potential for undesirable side effects arising from a high maximum plasma concentration (Cₘₐₓ) of the agent or therapeutically active metabolite thereof.

Relatively few agents having solubility not less than about 10 mg/ml are non-ionizable compounds. Most are compounds that exist as free acid or free base form and are present in such form, or more commonly in the form of a pharmaceutically acceptable salt, in a composition of the invention. Solubility of preferred agents is not less than about 50 mg/ml, more preferably not less than about 100 mg/ml. For purposes of the present invention, agents classified in the United States Pharmacopeia, 24th edition (2000) (USP 24) as “very soluble” or “freely soluble” are considered to have solubility not less than about 100 mg/ml, and together with agents classified in USP 24 as “soluble” or “sparingly soluble” are considered to have solubility not less than about 10 mg/ml.

Active pharmaceutical agents useful herein can be of any therapeutic category, for example any of the therapeutic categories listed in The Merck Index, 13th edition (2001). A partial list of agents useful herein is given below for illustration, noting that where one or more salts of an agent are listed, other pharmaceutically acceptable salts having solubility not less than about 10 mg/ml (“analogous salts”) can be substituted:

abacavir sulfate

acarbose

acebutolol hydrochloride

acetylsalicylic acid calcium salt

acyclovir sodium

albuterol sulfate

alendronate sodium

alfentanil hydrochloride

almotriptan maleate

alosetron hydrochloride

amantadine hydrochloride

amdinocillin

aminolevulinic acid hydrochloride

aminophylline

p-aminosalicylate calcium, sodium and potassium salts

amitryptiline hydrochloride

amlodipine acetate, hydrochloride and mesylate salts

amphetamine phosphate and sulfate salts

arbutamine

atenolol

atropine sulfate

azlocillin sodium

balsalazide disodium

benazipril hydrochloride

benztropine mesylate

bethanechol chloride

bisoprolol fumarate

brompheniramine maleate

bupropion hydrochloride

caffeine and caffeine citrate

capecitabine

captopril

carbenicillin disodium

carbetapentane citrate

carbinoxamine maleate

cefaclor

cefmetazole sodium

cefodizime disodium

ceftezole sodium

ceftiofur sodium

ceftriaxone disodium

cefuroxime sodium

cefuzonam sodium

cetirizine hydrochloride

cevimeline hydrochloride

chlordiazepoxide hydrochloride

chloroquine phosphate

chlorpheniramine maleate

chlorpromazine hydrochloride

cilastatin sodium

cimetidine hydrochloride

clindamycin hydrochloride

clindamycin phosphate

clomipramine hydrochloride

clonidine hydrochloride

clorazepate dipotassium

codeine hydrochloride and sulfate salts

codeine phosphate

cyclobenzaprine hydrochloride

cyproheptadine hydrochloride

cysteamine hydrochloride

daunorubicin hydrochloride

desipramine hydrochloride

dexamethasone 21-phosphate disodium

dextromethorphan hydrobromide

dibekacin

diclofenac potassium

dicloxacillin sodium

dicyclomine hydrochloride

didanosine

dihydrocodeine

diltiazem hydrochloride

diphenhydramine hydrochloride

disulfiram

dolasetron mesylate

donepezil hydrochloride

dopamine hydrochloride

dorzolamide hydrochloride

doxepin hydrochloride

doxycycline hyclate

eletriptan hemisulfate

enalapril maleate

epinastine hydrochloride

erythromycin glucoheptonate and lactobionate salts

ethosuximide

etidronic acid and disodium salt

etoperidone hydrochloride

fadrozole hydrochloride

famciclovir

fentanyl citrate

flucytosine

fludarabine phosphate

fluoxetine hydrochloride

fluvastatin sodium

fosfomycin and fosfomycin tromethamine

fosfosal

fosinopril sodium

fosphenytoin disodium

frovatriptan succinate

gabapentin

gatifloxacin

glycopyrrolate

granisetron hydrochloride

guaifenesin

haloperidol hydrochloride

homatropine methylbromide

hydralazine hydrochloride

hydrocodone bitartrate and hydrochloride salts

hydromorphone hydrochloride

hydroxychloroquine sulfate

hydroxyzine dihydrochloride

hyoscyamine hydrobromide

imatinib mesylate

imipramine hydrochloride

incadronate disodium

indinavir sulfate

isoniazid

isoxsuprine hydrochloride

ketorolac tromethamine

labetalol hydrochloride

lamivudine

levamisole hydrochloride

levetiracetam

lidocaine hydrochloride

lisinopril

losartan potassium

mafenide acetate

mecamylamine hydrochloride

medetomidine hydrochloride

meglutol

meperidine hydrochloride

metaraminol bitartrate

metformin hydrochloride

methadone hydrochloride

methamphetamine hydrochloride

methenamine

methimazole

methscopolamine bromide

methyldopa

methylphenidate hydrochloride

methylprednisolone 21-succinate sodium

metoclopramide

metoprolol succinate

metralindole hydrochloride

mexiletine hydrochloride

mezlocillin sodium

midazolam hydrochloride

midodrine hydrochloride

miglitol

mizoribine

moexipril hydrochloride

molindone hydrochloride

montelukast sodium

morphine hydrochloride

morphine sulfate

morpholine salicylate

nalmefene hydrochloride

naloxone hydrochloride

naproxen sodium

naratriptan hydrochloride

nedocromil disodium

neostigmine bromide and methylsulfate salts

nicotine bitartrate, salicylate and sulfate salts

nitrofurantoin

nizatidine

nortriptyline hydrochloride

ofloxacin

olsalazine sodium

ondansetron hydrochloride

orphenadrine hydrochloride

oxybutynin hydrochloride

oxycodone hydrochloride

pantoprazole sodium

parecoxib sodium

pemirolast potassium

penicillamine

penicillin G sodium and potassium salts

penicillin V potassium

pentamidine isethionate

pentobarbital sodium

pentosan polysulfate sodium

pentoxifylline

perindopril erbumine

phendimetrazine tartrate

phenelzine sulfate

phenoxybenzamine hydrochloride

phentermine hydrochloride

phenylephrine hydrochloride

phenylpropanolamine hydrochloride

phenyloin sodium

phenyltoloxamine citrate

pidotimod

pilocarpine hydrochloride

piperacillin sodium

pirenzepine dihydrochloride

pramipexole dihydrochloride

pravastatin sodium

prednisolone sodium phosphate

procainamide hydrochloride

procarbazine hydrochloride

procodazole

promethazine hydrochloride

propacetamol hydrochloride

propoxyphene hydrochloride

propranolol hydrochloride

protriptyline hydrochloride

pseudoephedrine hydrochloride

pyridostigmine bromide

quetiapine hemifumarate

quinapril hydrochloride

quinidine gluconate

quinine bisulfate

rabeprazole sodium

raltitrexed

ramosetron hydrochloride

ranitidine hydrochloride

reboxetine mesylate and succinate salts

ribavirin

rimantadine hydrochloride

risedronate sodium

rivastigmine tartrate

rizatriptan benzoate

ropinirole hydrochloride

scopolamine hydrobromide

selegiline hydrochloride

sotalol hydrochloride

stavudine

sulbenicillin disodium

sulfacetamide sodium

sumanirole maleate

sumatriptan succinate

tacrine hydrochloride

taurolidine

terazosin hydrochloride

tetracycline hydrochloride

theobromine sodium acetate and theobromine sodium salicylate

theophylline ethanolamine and theophylline isopropanolamine

theophylline sodium acetate and theophylline sodium glycinate

thioridazine hydrochloride

thyroxine sodium

ticlopidine hydrochloride

timolol maleate

tolterodine tartrate

tramadol hydrochloride

trientine hydrochloride

trifluoperazine dihydrochloride

valacyclovir hydrochloride

valganciclovir hydrochloride

valproate sodium

venlafaxine hydrochloride

verapamil hydrochloride

warfarin sodium

zolmitriptan

zolpidem hemitartrate

Other agents useful herein include N-[5-(1,4-diazepan-1-yl)-2-[(3-fluorophenyl)sulfonyl]phenyl]acetamide, N-[(3R)-1-azabicyclo[2.2.2]oct-3-yl]furo[2,3-c]pyridine-5-carboxamide and salts thereof.

It will be understood that mention of an active pharmaceutical agent herein embraces racemates, enantiomers, polymorphs, hydrates and solvates thereof.

The present invention is especially suitable for highly potent drugs and prodrugs, i.e., those that are therapeutically effective in low daily dosage amounts, for example not greater than about 100 mg/day, especially not greater than about 50 mg/day, more especially not greater than about 25 mg/day, even more especially not greater than about 10 mg/day, and most especially not greater than about 5 mg/day.

In one embodiment the active pharmaceutical agent has therapeutic effect on the central nervous system (CNS). Such agents, herein referred to as “CNS agents”, are useful in treatment or prevention of CNS disorders, and include without limitation anticonvulsant, antidepressant, antidyskinetic, antiepileptic, antimanic, antimigraine, antimuscarinic, antiobsessional, antiparkinsonian, antipsychotic, antispasmodic, anxiolytic, cholinergic, CNS stimulant, dopamine receptor agonist, dopamine receptor antagonist, hypnotic, monoamine oxidase inhibitor, neuroleptic, neuroprotective, NMDA receptor antagonist, nootropic, prolactin inhibitor, sedative, selective serotonin reuptake inhibitor (SSRI), selective noradrenaline reuptake inhibitor (SNRI), serenic, serotonin receptor agonist, serotonin receptor antagonist and tranquilizer agents.

Illustrative CNS agents useful herein include salts of sumanirole, reboxetine and pramipexole.

Sumanirole is used preferably in the form of its R-enantiomer, (R)-5,6-dihydro-5-(methylamino)-4H-imidazo[4,5-ij]-quinolin-2(1H)-one (III) and can be substituted by its thione counterpart (R)-5,6-dihydro-5-(methylamino)-4H-imidazo[4,5-ij]-quinoline-2(1H)-thione (IV).

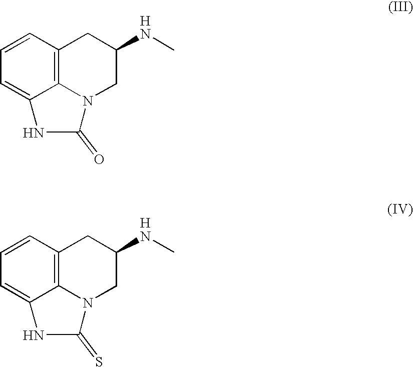

In the case of either compound (III) or (IV), suitable salts include hydrochloride, hydrobromide, hydroiodide, sulfate, phosphate, acetate, propionate, lactate, maleate, malate, succinate, tartrate, cyclohexanesulfamate, mesylate (methanesulfonate), esylate (ethanesulfonate), besylate (benzenesulfonate) and tosylate (p-toluenesulfonate) salts. The maleate salt is preferred.

Sumanirole compositions of the invention are suitable for administration no more than twice daily, preferably no more than once daily. Such compositions are useful in treatment of any CNS condition or disorder for which sumanirole has therapeutic utility, but especially Parkinson's disease and complications associated therewith.

Reboxetine (II) can be used in the form of a racemic mixture comprising two or more of (R,R)-reboxetine, (S,S)-reboxetine, (R,S)-reboxetine and (S,R)-reboxetine, or in the form of any of these enantiomers alone. Preferably (S,S)-reboxetine is used.

Suitable salts of reboxetine include hydrochloride, hydrobromide, hydroiodide, sulfate, phosphate, acetate, propionate, lactate, maleate, malate, succinate, fumarate, tartrate, cyclohexanesulfamate, mesylate, esylate, besylate and tosylate salts. In the case of a reboxetine racemate, the mesylate salt is preferred. In the case of (S,S)-reboxetine, the succinate and fumarate salts, more especially the succinate salt, are preferred.

Reboxetine and (S,S)-reboxetine compositions of the invention are preferably suitable for administration no more than once daily. Such compositions are useful in treatment of any CNS condition or disorder for which reboxetine and enantiomers thereof have therapeutic utility, but especially depressive illness and neuropathic pain, including postherpetic neuralgia and diabetic neuropathy.

Pramipexole (I) is used preferably in the form of its S-enantiomer, (S)-2-amino-4,5,6,7-tetrahydro-6-(propylamino)-benzothiazole. A preferred salt of pramipexole is the dihydrochloride salt, most preferably in the form of the monohydrate.

Pramipexole compositions of the invention are preferably suitable for administration no more than once daily. Such compositions are useful in treatment of any CNS condition or disorder for which pramipexole has therapeutic utility, but especially Parkinson's disease and complications associated therewith.

All active pharmaceutical agents useful herein can be prepared by processes known per se, including processes disclosed in patents and other literature pertaining to specific agents of interest.

The amount of the active pharmaceutical agent present in a composition of the invention depends on the potency of the agent, but is sufficient to provide a daily dose in one to a small plurality, for example one to about 4, of tablets to be administered at one time. Preferably the full daily dose is delivered in a single tablet. In most cases the amount of the agent per tablet is about 0.1 to about 200 mg, preferably about 0.2 to about 100 mg. Expressed as percentage by weight of the composition, the amount of the agent is typically about 0.01% to about 25%, preferably about 0.05% to about 20%. In the case of an agent that is a salt, amounts of agent herein are expressed as free acid or free base equivalent amounts, unless otherwise specified.

Illustratively in the case of sumanirole, an amount of about 0.5 to about 25 mg per tablet, or about 0.1% to about 15% by weight of the composition, will generally be suitable. Specific dosage amounts per tablet contemplated herein include 0.5, 1, 2, 4, 8, 12 and 24 mg sumanirole in the form of sumanirole maleate.

In the case of pramipexole, an amount of about 0.1 to about 10 mg per tablet, or about 0.05% to about 5% by weight of the composition, will generally be suitable. Preferably an amount of about 0.2 to about 6 mg, more preferably an amount of about 0.3 to about 5 mg, pramipexole per tablet is present. Specific dosage amounts per tablet contemplated herein include 0.375, 0.5, 0.75, 1.0, 1.5, 3.0 and 4.5 mg pramipexole dihydrochloride monohydrate.

In the case of reboxetine or (S,S)-reboxetine, an amount of about 0.2 to about 15 mg per tablet, or about 0.1% to about 10% by weight of the composition, will generally be suitable. Preferably an amount of about 1 to about 12 mg reboxetine or (S,S)-reboxetine per tablet is present. Specific dosage amounts per tablet contemplated herein include 1, 2, 4, 6, 8 and 12 mg reboxetine in the form of its mesylate salt or (S,S)-reboxetine in the form of its succinate salt.

A composition of the present invention comprises an active pharmaceutical agent as defined above, dispersed in a matrix comprising a hydrophilic polymer and a starch having a tensile strength of at least about 0.15 kN cm⁻² at a solid fraction representative of the tablet, for example about 0.75 to about 0.85, illustratively 0.8.

Hydrophilic polymers useful herein are pharmaceutically acceptable polymeric materials having a sufficient number and distribution of hydrophilic substituents such as hydroxy and carboxy groups to impart hydrophilic properties to the polymer as a whole. Suitable hydrophilic polymers include, without limitation, methylcellulose, HPMC (hypromellose), carmellose (carboxymethylcellulose) sodium and carbomer (polyacrylic acid). More than one such polymer can optionally be used.

HPMC is a preferred hydrophilic polymer. Various types and grades of HPMC are available. In one embodiment HPMC type 2208, preferably meeting specifications set forth in a standard pharmacopeia such as USP 24, is used. HPMC type 2208 contains 19-24% by weight methoxy and 4-12% by weight hydroxypropoxy substituents. Especially suitable HPMCs have nominal viscosity ranging from about 100 to about 10,000 mPa s; illustratively a suitable HPMC type 2208 is one having a nominal viscosity of about 4,000, with a measured viscosity of about 3,000 to about 5,600 mPa S. Such an HPMC is available, for example, as Methocel® K4MP from Dow Chemical Co., and substantially equivalent products are available from other manufacturers.

The amount of hydrophilic polymer in the composition depends on the particular polymer selected, on the active pharmaceutical agent and on the desired sustained release profile. Typically, however, the hydrophilic polymer is included in an amount of about 20% to about 70%, preferably about 30% to about 60% and more preferably about 35% to about 50%, by weight of the composition. In the illustrative case of HPMC type 2208, a suitable amount will generally be found in the range from about 30% to about 60%, preferably about 35% to about 50%, for example about 40%, by weight of the composition.

It is believed, without being bound by theory, that the hydrophilic polymer functions to provide extended or sustained release of the active pharmaceutical agent, for example by gradual dissolution or erosion of the polymer in the gastrointestinal tract.

Starches useful herein include starches from any suitable botanical source, for example corn, wheat, rice, tapioca, potato, etc. Preferred starches have a relatively high ratio of amylose to amylopectin, containing for example at least about 20%, more preferably at least about 25%, amylose. Especially preferred is pregelatinized starch, which is a type of modified starch that has been processed to render the starch more flowable and directly compressible. Partially or wholly pregelatinized starches can be used.

It is believed, without being bound by theory, that the primary function of the starch in a composition of the invention is as a binding agent. A starch meeting the tensile strength criterion defined herein can be referred to as a “super binder”.

The amount of starch in the composition is typically higher than is conventionally present as a binder in tablet formulations. Suitable amounts will generally be found in the range of about 25% to about 75% by weight. Preferably the amount of starch is about 40% to about 70%, more preferably about 45% to about 65%, for example about 50%, by weight of the composition.

Tensile strength of a starch sample can be measured by any suitable test. Illustrative test procedures are described by Hiestand & Smith (1984), Powder Technology 38, 145-159, and by Hiestand & Smith (1991), International Journal of Pharmaceutics 67, 231-246, these articles being incorporated herein by reference.

An example of a tensile strength test that can be used (herein referred to as a “triaxial tensile strength test”) requires preparation of a series of compacts of the starch sample, followed by determination of tensile strength of the compacts using a computerized multifunction tablet tester (MTT). The compacts are prepared with various degrees of compression force to provide compacts having a range of solid fraction. As a sustained release tablet formulation typically has a solid fraction of about 0.8, it is useful to prepare compacts approximating such a solid fraction.

Absolute density of the starch sample can be determined using a helium-air pycnometer.

A computer-controlled triaxial tablet press is used to prepare the compacts. Voltage output from the punch and die load cells of the tablet press are first zeroed. The punch and die are lubricated with magnesium stearate powder and the die assembly is placed in the press. Compression and decompression parameters are selected on the computer. The desired amount of starch to be compacted is weighed and poured into the die cavity. The resulting powder bed is leveled with a spatula. The punch is inserted into the die and the computer-controlled compression/decompression cycle is started.

Just prior to the end of the compression phase, thickness of the compact as measured by LVDT is recorded. At the end of the compression phase, the final compression force as measured by voltage of the punch load cell is recorded.

At the end of the decompression phase, the punch and die rams are retracted. The compact is removed from the die and inspected for defects, such as cracking or sticking. Cracking can be reduced by increasing decompression time. If the compact is free of defects, its length, width, thickness and weight are measured to enable calculation of apparent density. Solid fraction is calculated by dividing absolute density by apparent density.

In preparation of the MTT for tensile strength determination, a suitable software program is run. The platen is screwed to the load cell of the MTT and the tensile strength assembly is slid into the MTT opposite the platen. The load cell signal is monitored via the computer and the zero offset on the signal conditioner is adjusted to provide a positive baseline voltage as close as possible to zero. A forward velocity is selected that will generate a time constant of approximately 15 seconds (usually the velocity selected will be about 0.8 to about 1.2 mm s⁻¹).

The compact to be tested is placed in the holder of the tensile strength assembly. The motor is initiated via the computer, driving the platen toward the compact until the surface of the compact is detected, and stopping the platen a few millimeters from the compact. The oscilloscope is triggered, to record the force applied to the compact, and the motor is restarted. The platen is driven into the compact until a crack is detected, either by sight or by sound, and the motor is immediately reversed.

Peak force is recorded from the oscilloscope trace. Tensile strength is calculated from the peak force using appropriate computer software.

From several runs using compacts at a range of solid fractions around 0.8, data are plotted and tensile strength at a solid fraction of 0.8 is estimated. If the tensile strength at a solid fraction of 0.8 is about 0.15 kN cm⁻² or greater, the starch sample is deemed to be suitable for use in preparing a composition according to the invention.

It has now surprisingly been discovered that a much simpler test, one that is more amenable to implementation in a manufacturing setting, can be used to estimate tensile strength of a starch sample, in particular to determine whether the starch sample has a tensile strength of at least about 0.15 kN cm⁻² at a solid fraction representative of a desired sustained-release tablet.

According to this test, compacts of the starch sample are prepared on a standard automated tablet press under a range of compression forces. For example, a Carver press (e.g., Model 3888.1DT0000) fitted with flat-faced tooling of suitable diameter (e.g., 10/32 inch or about 0.7 cm for a 300 mg compact), operated at compression forces of about 4 to about 16 kN (about 900 to about 3600 lbf) for a dwell time of at least about 4 seconds has been found to give satisfactory results. Illustratively, such compacts can be prepared at 1000, 1500, 2000 and 3000 lbf (4.45, 6.67, 8.90 and 13.34 kN). Preferably a dwell time of at least about 10 seconds, more preferably at least about 30 seconds, still more preferably at least about 60 seconds, is used. Illustratively, a dwell time of 90 seconds has been found to give satisfactory results. Weight, diameter and thickness of each compact are measured accurately (alternatively, diameter can be assumed to equal that of the tooling) to enable calculation of apparent density and hence solid fraction, absolute density having been measured as described above, for example by helium-air pycnometry.

Hardness of each compact thus prepared is then determined by any suitable tablet hardness test, for example using a Key HT 500 hardness tester. Hardness is a measure of the force required to cause crushing of the compact, and is typically expressed in units such as kiloponds (kp) or Strong-Cobb units (SCU). A hardness of about 10.2 kp or about 14.4 SCU corresponds to a force of 0.1 kN.

For present purposes it is considered that crushing strength of the compact is equivalent to tensile strength. Thus tensile strength (σ_{T}, in kN cm⁻²) can be calculated from the equation

σ_{T}=2F/πDH

where F is the force required to cause crushing (in kN), D is diameter of the compact (in cm) and H is thickness of the compact (in cm). For example, a compact of diameter 0.7 cm and thickness 0.4 cm having a hardness of 20 SCU (equivalent to a force of 0.139 kN) has a calculated tensile strength of 0.316 kN cm⁻².

The relationship between tensile strength and solid fraction is next established for the starch sample. This can be done by plotting data for tensile strength and solid fraction on a graph (solid fraction tends to increase with increasing compression force during preparation of the compact) or by performing a regression analysis. From that relationship, tensile strength at a standardized value of solid fraction can be estimated. The standardized value selected is one that is representative of the solid fraction of a desired sustained-release tablet, e.g., 0.8.

Where the material of the compact is pregelatinized starch, it has been found that tensile strength as determined in a simple test as described immediately above is surprisingly close to a “true” tensile strength measurement as determined by the triaxial tensile strength test method previously described, which in turn is essentially similar to methods known in the art such as that disclosed by Hiestand & Smith (1984), op. cit.

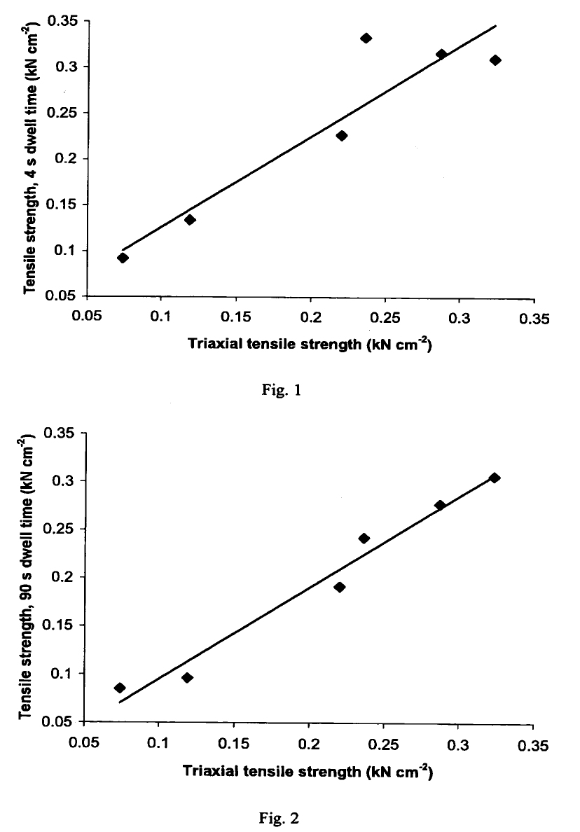

It has also been found that a longer dwell time (e.g., 90 seconds) in the test method of the present invention gives a better correlation with triaxial tensile strength than a very short dwell time (e.g., 4 seconds). See Example 1 below and FIGS. 1 and 2.

Thus, according to one embodiment of the invention, a method is provided for determining suitability of a starch for use in a sustained-release orally deliverable tablet comprising an active pharmaceutical agent having solubility not less than about 10 mg/ml, dispersed in a matrix comprising a hydrophilic polymer and a starch. The method comprises the steps of
(a) preparing compacts of a starch sample on an automated tablet press at a range of compression forces applied for a dwell time of at least about 4 seconds;
(b) measuring hardness of each compact, expressed as the force required to cause crushing of the compact;
(c) determining solid fraction of each compact;
(d) calculating tensile strength σ_{T} of each compact from the equation

σ_{T}=2F/πDH

where F is the force required to cause crushing, D is diameter of the compact and H is thickness of the compact;
(e) establishing relationship of tensile strength to solid fraction of the compacts, for example by plotting these parameters on a graph and/or by performing a regression analysis; and
(f) using the relationship established in step (e), estimating tensile strength at a solid fraction representative of a desired sustained-release tablet; for example a solid fraction of 0.8.

If tensile strength of the starch as so estimated is at least about 0.15 kN cm⁻², the starch is deemed suitable for use.

An especially preferred starch has a tensile strength of at least about 0.175 kN cm⁻², even more preferably at least about 0.2 kN cm⁻², at a solid fraction representative of a desired sustained-release tablet.

Even among commercially available pregelatinized starches, the preferred type of starch for use in a composition of the invention, considerable variation exists in tensile strength. Pregelatinized starches not meeting the tensile strength criterion established herein are not readily identified without testing, for example by a method as disclosed above. Such pregelatinized starches are generally unsuitable for commercial-scale manufacture of a sustained-release matrix tablet formulation of a water-soluble drug or prodrug, because of a problem as set forth immediately below.

An uncoated tablet, or a tablet core prior to coating, comprising starch and a hydrophilic polymer acting as a matrix for a water-soluble drug or prodrug requires to have a certain minimum hardness in order to be able to resist breakage and/or attrition due to mechanical stresses imposed during a high-speed tableting operation (including all steps up to and including filling of the tablets into containers). The minimum acceptable hardness will depend on a number of factors, including the severity of the mechanical stresses, but is typically at least about 20 SCU, preferably at least about 22 SCU, more preferably at least about 24 SCU (about 17 kp).

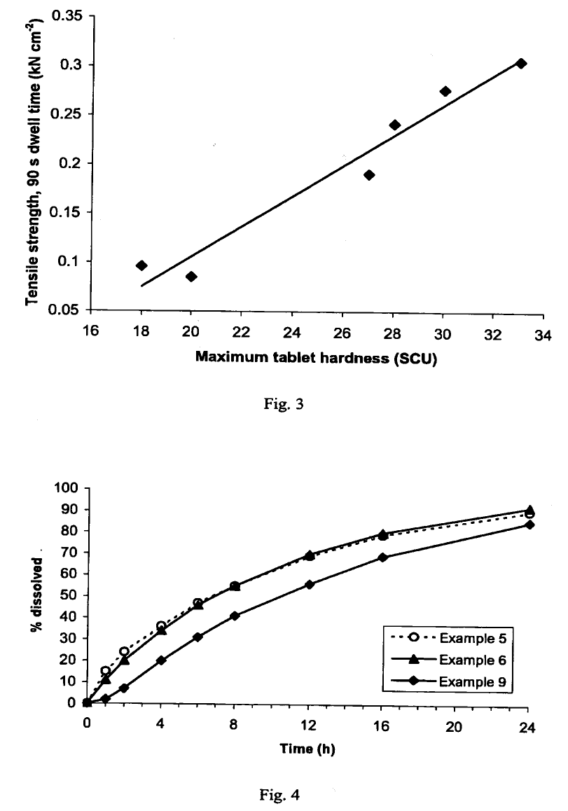

Hardness can be increased by increasing the compression force applied by the tablet press, but only up to a certain level. At least in the case of tablets as described herein, above a certain compression force, further increases in compression force give little or no further increase in tablet hardness. There is, in other words, a maximum hardness achievable by compression of a particular starch/hydrophilic polymer/active agent composition. A starch providing a maximum hardness inadequate to withstand the mechanical stresses of a high-speed tableting operation is unsuitable for the present purpose. As shown in FIG. 3, certain pregelatinized starches have been found to provide a maximum hardness of 20 SCU or less; these are now identified as starches having low tensile strength (0.1 kN cm⁻² or less according to the test method of the invention utilizing a dwell time of 90 seconds).

Even if a maximum hardness of at least about 20 SCU is achievable, with a starch of low tensile strength it may be achievable only by use of extremely high compression forces. A requirement for such forces reduces speed and efficiency and increases cost of a tableting operation and is undesirable for these reasons.

Where tablets are to be subjected to an additional process step after compression, in particular a coating step, exposure to mechanical stresses is greatly increased. According to a preferred embodiment, therefore, the sustained-release tablet of the invention further comprises a coating.

For drugs and prodrugs of high water solubility as specified herein, a hydrophilic polymer matrix is often inadequate to provide sustained release of sufficiently long duration to permit once daily administration. It is believed that such drugs are readily leached out of the hydrophilic matrix when contacted by an aqueous medium such as gastrointestinal fluid. It is therefore desirable to further slow the process of drug release by providing a release-controlling coating around the tablet. Such a coating typically comprises a hydrophobic or water-insoluble polymer component such as ethylcellulose together with a hydrophilic or water-soluble pore-forming component such as HPMC.

Where a starch is used having a tensile strength of at least about 0.15 kN cm⁻², preferably at least about 0.175 kN cm⁻², more preferably at least about 0.2 kN cm⁻², at a solid fraction representative of the tablet (e.g., about 0.75 to about 0.85), the composition is found to be especially suited to a high-speed tableting operation that includes a step of coating the tablet with a release-controlling layer.

Alternatives to ethylcellulose and HPMC as components of a release coating layer include other cellulosic polymers (e.g., methylcellulose, hydroxypropylcellulose, hydroxyethylcellulose, carboxymethylcellulose sodium, cellulose esters such as cellulose acetate, etc.), polyvinyl acetate, polyvinyl pyrrolidone, polymers and copolymers of acrylic acid and methacrylic acid and esters thereof, polyethylene glycol carrageenan and other gums, and the like.

A release-controlling layer, if present, typically constitutes about 1% to about 15%, preferably about 2.5% to about 10%, by weight of the tablet as a whole. The hydrophobic or water-insoluble component, preferably comprising ethylcellulose, typically constitutes about 1% to about 10%, preferably about 2% to about 7%, by weight of the tablet as a whole. The pore-forming component, preferably comprising HPMC, is typically present in an amount of about 5% to about 50%, preferably about 10% to about 40%, by weight of the water-insoluble or hydrophobic component.

The coating, if present, can optionally contain additional pharmaceutically acceptable excipients such as plasticizers, dyes, etc.

Illustratively, a release-controlling layer in an amount of about 2.5% to about 5% by weight of the tablet core (i.e., the tablet weight excluding the coating) comprises an ethylcellulose-based material (e.g., Surelease® of Colorcon) and an HPMC-based pore-forming material (e.g., Opadry® of Colorcon) in a weight ratio of about 3:1 to about 4:1.

A release-controlling layer or coating should be applied at as uniform a thickness as possible to provide optimum control of release rate of the active pharmaceutical agent.

Alternatively or in addition, the sustained-release tablet of the invention comprises a nonfunctional coating. A nonfunctional coating can comprise a polymer component, for example HPMC, optionally with other ingredients, for example one or more plasticizers, colorants, etc. The term “nonfunctional” in the present context means having no substantial effect on release properties of the tablet, and does not imply that the coating serves no useful purpose. For example, such a coating can impart a distinctive appearance to the tablet, provide protection against attrition during packaging and transportation, improve ease of swallowing, and/or have other benefits. A nonfunctional coating should be applied in an amount sufficient to provide complete coverage of the tablet. Typically an amount of about 1% to about 10%, more typically an amount of about 2.5% to about 5%, by weight of the tablet as a whole, will be found suitable.

Uncoated tablets and cores of coated tablets of the invention optionally contain one or more pharmaceutically acceptable excipients in addition to the starch and hydrophilic polymer components described above. Such excipients include without limitation glidants and lubricants. Other conventional excipients known in the art can also be included.

A glidant can be used to improve powder flow properties prior to and during tableting and to reduce caking. Suitable glidants include colloidal silicon dioxide, magnesium trisilicate, powdered cellulose, starch, talc, tribasic calcium phosphate and the like. In one embodiment, colloidal silicon dioxide is included as a glidant in an amount up to about 2%, preferably about 0.2% to about 0.6%, by weight of the tablet.

A lubricant can be used to enhance release of a tablet from apparatus on which it is formed, for example by preventing adherence to the face of an upper punch (“picking”) or lower punch (“sticking”). Suitable lubricants include magnesium stearate, calcium stearate, canola oil, glyceryl palmitostearate, hydrogenated vegetable oil, magnesium oxide, mineral oil, poloxamer, polyethylene glycol, polyvinyl alcohol sodium benzoate, sodium lauryl sulfate, sodium stearyl fumarate, stearic acid, talc, hydrogenated vegetable oil, zinc stearate and the like. In one embodiment, magnesium stearate is included as a lubricant in an amount of about 0.1% to about 1.5%, preferably about 0.3% to about 1%, by weight of the tablet.

Tablets can be of any suitable size and shape, for example round, oval, polygonal or pillow-shaped, and optionally bear nonfunctional surface markings. Especially in the case of coated tablets they are preferably designed to be swallowed whole and are therefore typically not provided with a breaking score. Tablets of the invention can be packaged in a container, accompanied by a package insert providing pertinent information such as, for example, dosage and administration information, contraindications, precautions, drug interactions and adverse reactions.

There is also provided a method of treatment of a subject having a condition or disorder for which an active pharmaceutical agent having solubility not less than about 10 mg/ml is indicated, the method comprising orally administering to the subject a sustained-release pharmaceutical composition in a form of a tablet comprising the agent dispersed in a matrix comprising a hydrophilic polymer and a starch having a tensile strength of at least about 0.15 kN cm⁻² at a solid fraction representative of the tablet. Preferably the composition is administered no more than once daily.

In one embodiment, the condition or disorder is a CNS condition or disorder and the agent is a CNS agent as defined herein. CNS conditions and disorders include those having a neurologic and/or a psychiatric component.

Illustrative CNS conditions and disorders treatable by the method of the invention include, for example, personality disorders including paranoid, schizoid, schizotypal, bipolar, histrionic, delusional, narcissistic, emotionally unstable, psychopathic and sociopathic personality disorders; habit and impulse disorders including pathological gambling, stealing, trichotillomania, etc.; obsessive-compulsive disorder; passive-aggressive disorder; acute and transient psychotic disorders; psychotic depression; schizoaffective disorder; hypochondria; cyclothymia; dysthymia; manic-depressive illness; major depressive disorder; treatment-resistant depression; adult and childhood onset schizophrenias; drug dependence including harmful use and abuse of, addiction to or dependence on opioids, narcotics, barbiturates, alcohol, benzodiazepines, amphetamines, cocaine, cannabinoids, hallucinogens, stimulants, nicotine (tobacco) and solvents; withdrawal states and mood and psychotic disorders related to such dependence; sexual dysfunction including hypoactive sexual desire disorder, sexual aversion or avoidance and erectile dysfunction; gender identity disorders; sexual preference disorders; general anxiety disorder; social anxiety disorder; mixed anxiety and depressive disorder; attention deficit hyperactivity disorder (ADHD) and depression and anxiety associated therewith; depression, anxiety, emotional dysregulation and behavioral disturbances associated with mental retardation; developmental disorders including autism, Asperger's syndrome and Rett's syndrome; childhood conduct and attachment disorders; premenstrual dysphoric disorder; postpartum depression; phobias including social phobias, agoraphobia and specific phobias related for example to hospitals, injections, venesection, etc.; posttraumatic stress disorder; dissociative disorder; Briquet's syndrome; affective disorders including depression, bipolar affective disorder and recurrent depressive disorder; organic mood, anxiety and emotionally labile disorders resulting for example from brain damage or dysfunction arising from head injury, intracranial masses, stroke, etc.; chronic fatigue; stress-induced psychotic episodes; dementia including presenile dementia, Pick's disease, vascular dementia, multi-infarct dementia, Alzheimer's disease, dementia associated with Creutzfeldt-Jakob disease and HIV-related dementia; other neurodegenerative disorders including Parkinson's disease and Huntington's disease; suicidal behavior; eating disorders including anorexia, bulimia and binge eating disorder; adjustment disorders; somatization disorder; somatoform autonomic dysfunction; somatoform pain disorder; panic attacks; panic disorder; amnesia; neuropathic pain; fibromyalgia; migraine; epilepsy; tinnitus; enuresis; sleep disorders including insomnia, hypersomnia, narcolepsy, nightmares and night terrors; delirium; postconcussion syndrome; multiple sclerosis; tremors; muscular spasms; restless legs syndrome; Lennox-Gastaut syndrome; motor and vocal tic disorders; Tourette's syndrome; supranuclear palsy; Shy-Drager syndrome; trigeminal neuralgia; Bell's palsy; motor neuron diseases such as amyotrophic lateral sclerosis; and psychosomatic and psychosocial conditions associated with non-CNS diseases such as diabetes, inflammatory disease, infertility, allergies, psoriasis, asthma, hypertension, overactive bladder, thyroid disorders, obesity, immune disorders and cancer.

In a particular embodiment, the condition or disorder is one that is responsive to dopamine D₂ receptor agonist therapy or to SNRI therapy, and the active pharmaceutical agent is a dopamine D₂ receptor agonist or SNRI or prodrug thereof. Presently preferred dopamine D₂ receptor agonists for use according to the method of the invention include salts of pramipexole and sumanirole. Such dopamine D₂ receptor agonists are particularly useful in treatment of Parkinson's disease. Presently preferred SNRIs for use according to the method of the invention include salts of reboxetine and (S,S)-reboxetine. Such SNRIs are particularly useful in treatment of depressive illness and neuropathic pain, including postherpetic neuralgia and diabetic neuropathy.

Illustratively in the case of sumanirole, suitable daily dosage amounts include 0.5, 1, 2, 4, 8, 12 and 24 mg sumanirole in the form of sumanirole maleate. In the case of pramipexole, suitable daily dosage amounts include 0.375, 0.5, 0.75, 1.0, 1.5, 3.0 and 4.5 mg pramipexole dihydro chloride monohydrate. In the case of reboxetine or (S,S)-reboxetine, suitable daily dosage amounts include 1, 2, 4, 6, 8 and 12 mg reboxetine in the form of its mesylate salt or (S,S)-reboxetine in the form of its succinate salt.

In a further embodiment, a composition of the invention is administered in combination therapy with one or more additional drugs or prodrugs. The term “combination therapy” herein means a treatment regimen wherein the agent provided by the composition of the invention and a second agent are administered individually or together, sequentially or simultaneously, in such a way as to provide a beneficial effect from co-action of these therapeutic agents. Such beneficial effect can include, but is not limited to, pharmacokinetic or pharmacodynamic co-action of the therapeutic agents. Combination therapy can, for example, enable administration of a lower dose of one or both agents than would normally be administered during monotherapy, thus decreasing risk or incidence of adverse effects associated with higher doses. Alternatively, combination therapy can result in increased therapeutic effect at the normal dose of each agent in monotherapy. “Combination therapy” herein is not intended to encompass administration of two or more therapeutic agents as part of separate monotherapy regimens that incidentally and arbitrarily result in sequential or simultaneous treatment.

Compositions of the invention can be especially suited to combination therapies, particularly where the second agent is one that is, or can be, administered once daily. There are significant advantages in patient convenience and compliance where both components of a combination therapy can be administered at the same time and with the same frequency. This is especially true in the case of geriatric patients or those suffering memory impairment.

When administered simultaneously, the two components of the combination therapy can be administered in separate dosage forms or in coformulation, i.e., in a single dosage form. When administered sequentially or in separate dosage forms, the second agent can be administered by any suitable route and in any pharmaceutically acceptable dosage form, for example by a route and/or in a dosage form other than the present composition. In a preferred embodiment, both components of the combination therapy are formulated together in a single dosage form.

An illustrative combination therapy involves once daily administration of a composition of the invention comprising an SNRI, for example a salt of reboxetine or (S,S)-reboxetine, and once daily administration of an SSRI, for example fluoxetine, fluvoxamine, paroxetine or sertraline or a salt thereof. SNRI/SSRI combination therapies have been proposed, e.g., for treatment-resistant depression as disclosed by Forbes & Rogers (2003), Progress in Neurology and Psychiatry 7(2), 10-14; according to the present invention both components of such a combination therapy can be administered once daily, with concomitant improvement in patient compliance.

### EXAMPLES

### Example 1

Tensile strength of six commercially obtained lots of pregelatinized starch was determined using the triaxial tensile strength test procedure described hereinabove. Data for tensile strength at a solid fraction of 0.8 are presented in Table 1.

TABLE 1
Tensile strength of pregelatinized starch lots at a solid fraction of 0.8
(triaxial test procedure)

| Lot | Tensile strength (kN cm⁻²) |
|---|---|
| 1 | 0.323 |
| 2 | 0.220 |
| 3 | 0.074 |
| 4 | 0.119 |
| 5 | 0.287 |
| 6 | 0.236 |

A great variation in tensile strength of pregelatinized starches was observed, ranging from 0.074 to 0.323 kN cm⁻². Lots 3 and 4, exhibiting the lowest values of tensile strength, were from one manufacturer. Lots 1, 5 and 6, exhibiting the highest values of tensile strength, were from a second manufacturer. Lot 2, exhibiting an intermediate value of tensile strength, was from a third manufacturer.

### Example 2

Tensile strength of the same six lots of pregelatinized starch was determined by the following simplified test procedure.

Compacts of each starch lot were prepared on a Carver press, Model 3888.1DT0000 fitted with 10/32 inch (0.7 cm) flat-faced tooling, at compression forces of 1000, 1500, 2000 and 3000 lbf (4.45, 6.67, 8.90 and 13.34 kN), for a dwell time of 4 seconds or 90 seconds. Compacts of an additional three lots of pregelatinized starch (Lots 7, 8 and 9), from the same manufacturer as Lots 3 and 4, were prepared using a dwell time of 90 seconds only. Weight and thickness of each compact was measured (diameter being equal to that of the tooling) to enable calculation of apparent density. Absolute density of each starch lot was measured by helium-air pycnometry. Solid fraction was calculated as the ratio of apparent to absolute density.

Hardness (force required to cause crushing) of each compact was determined using a Key HT 500 hardness tester. Tensile strength was calculated from this force and dimensions of the compact, using the equation

σ_{T}=2F/πDH

as described hereinabove.

A regression analysis was performed to determine the relationship of tensile strength to solid fraction for each starch lot, and tensile strength at a standardized solid fraction of 0.8 was calculated. Data are presented in Table 2.

TABLE 2
Tensile strength of pregelatinized starch lots at a solid fraction of 0.8
(simplified test procedure of the invention)

Tensile strength
(kN cm⁻²)

| Lot | 4 s dwell time | 90 s dwell time |
|---|---|---|
| 1 | 0.310 | 0.306 |
| 2 | 0.227 | 0.191 |
| 3 | 0.092 | 0.085 |
| 4 | 0.134 | 0.096 |
| 5 | 0.316 | 0.277 |
| 6 | 0.333 | 0.242 |
| 7 | n.d. | 0.087 |
| 8 | n.d. | 0.088 |
| 9 | n.d. | 0.172 |

Correlation of tensile strength as measured in the simplified test using a 4 second dwell time (this Example) with tensile strength as measured by the triaxial test procedure of Example 1 is shown graphically in FIG. 1.

Correlation of tensile strength as measured in the simplified test using a 90 second dwell time (this Example) with tensile strength as measured by the triaxial test procedure of Example 1 is shown graphically in FIG. 2.

Both dwell times exhibited a strong correlation, but the correlation was especially close where the simplified test used a 90 second dwell time. It is concluded that the simplified test as herein described can be used to estimate tensile strength of a starch lot for the purpose of predicting whether that starch lot will be suitable for preparing a sustained-release tablet formulation of the present invention.

### Example 3

Sumanirole maleate sustained-release tablets were prepared having the compositions shown in Table 3. Tablet strength in mg is expressed as sumanirole base.

TABLE 3
Composition of sumanirole maleate tablets of Example 3

| | Tablet strength (mg) | | | | | | | |
|---|---|---|---|---|---|---|---|---|
| | 0.5 | 1 | 2 | 4 | 8 | 8 | 12 | 24 |
| Ingredient | Amount (% by weight) | | | | | | | |
| sumanirole | 0.23 | 0.45 | 0.9 | 1.8 | 3.6 | 3.6 | 5.4 | 10.9 |
| maleate | | | | | | | | |
| HPMC | 35.00 | 35.00 | 35.0 | 35.0 | 35.0 | 35.0 | 35.0 | 35.0 |
| type 2208, | | | | | | | | |
| 4000 mPa s | | | | | | | | |
| pregelatinized | 63.87 | 63.65 | 63.2 | 62.3 | 60.5 | 60.0 | 58.2 | 52.5 |
| starch | | | | | | | | |
| colloidal | 0.40 | 0.40 | 0.4 | 0.4 | 0.4 | 0.4 | 0.4 | 0.4 |
| silicon | | | | | | | | |
| dioxide | | | | | | | | |
| magnesium | 0.50 | 0.50 | 0.5 | 0.5 | 0.5 | 1.0 | 1.0 | 1.0 |
| stearate | | | | | | | | |

All ingredients except the lubricant (magnesium stearate) were screened to remove lumps and were blended thoroughly in a low-shear mixer operating at 24 rpm for 10-30 minutes. The lubricant was then screened into the mixer and the materials were blended for a further 2-5 minutes. The resulting lubricated mixture was compressed into 350 mg pillow-shaped tablets using a Kilian S100 tableting machine.

### Example 4

Tablets similar to those of Example 3 were prepared using pregelatinized starches of lots 1-6 as tested in Examples 1 and 2. Maximum hardness of the tablets obtainable with each pregelatinized starch lot was determined.

Maximum hardness was correlated with tensile strength of the pregelatinized starch lot used, as measured in the simplified test of Example 2 using a 90 second dwell time. Results are shown in FIG. 3. The correlation was substantially linear.

In subsequent tests, tablets of different hardness were used as cores for coating and were tested for resistance to erosion during a high-speed coating operation. Tablet cores having a hardness of at least about 24 SCU (about 17 kp) were found to have acceptable resistance to erosion. As shown in FIG. 3, this degree of hardness is achievable using pregelatinized starch having a tensile strength of at least about 0.175 kN cm⁻². Pregelatinized starches of Lots 3 and 4 were unsuitable, having tensile strength less than about 0.15 kN cm⁻² and providing tablets having a maximum hardness no greater than about 20 SCU (about 14 kp).

### Example 5

Pramipexole dihydrochloride sustained-release tablets were prepared having the compositions shown in Table 4.

TABLE 4
Composition of pramipexole dihydrochloride tablets of Example 5

| Ingredient | Amount (mg) | | | | | | | |
|---|---|---|---|---|---|---|---|---|
| pramipexole dihydrochloride | 0.375 | 0.75 | 1.5 | 3.0 | 4.5 | 0.375 | 0.375 | 4.5 |
| monohydrate | | | | | | | | |
| HPMC type 2208, 4000 mPa s | 140.0 | 140.0 | 140.0 | 140.0 | 140.0 | 70.0 | 157.5 | 157.5 |
| pregelatinized starch | 206.5 | 206.1 | 205.4 | 203.9 | 202.4 | 101.5 | 189.0 | 184.9 |
| colloidal silicon dioxide | 1.4 | 1.4 | 1.4 | 1.4 | 1.4 | 1.4 | 1.4 | 1.4 |
| magnesium stearate | 1.75 | 1.75 | 1.75 | 1.75 | 1.75 | 1.75 | 1.75 | 1.75 |
| total | 350 | 350 | 350 | 350 | 350 | 175 | 350 | 350 |

The tablets were prepared by the procedure described in Example 3, using pregelatinized starch having a tensile strength of at least about 0.175 kN cm⁻².

### Example 6

Coated sustained-release tablets of pramipexole dihydrochloride were prepared having the composition shown in Table 5.

TABLE 5
Composition of coated tablets of Example 6

| Ingredient | Amount (mg) |
|---|---|
| pramipexole dihydrochloride monohydrate | 0.375 |
| HPMC type 2208, 4000 mPa s | 140.0 |
| pregelatinized starch | 206.5 |
| colloidal silicon dioxide | 1.4 |
| magnesium stearate | 1.75 |
| total core | 350 |
| ethylcellulose-based coating material (Surelease ®) | 7.88 |
| HPMC-based coating material (Opadry ®) | 2.63 |
| total coating | 10.5 |

Tablet cores were prepared exactly as in Example 5, using pregelatinized starch having a tensile strength of at least about 0.175 kN cm⁻². A coating solution was prepared as follows. Opadry® HPMC-based material in an amount of 6.004 g was added to 106.682 g water and mixed for 45 minutes to provide an HPMC mixture. Next, 72.045 g Surelease® ethylcellulose-based material was added to the HPMC mixture and mixed for an additional 30 minutes to provide a coating solution.

The coating solution was applied to the tablet cores in an amount providing a 3% weight gain. The resulting coated tablets were cured using a 12 inch (about 30 cm) Vector LCDS or 24 inch (about 60 cm) Thomas Accela-Coata coating pan for about 15 minutes at a bed temperature of at least about 70° C. After curing, temperature was ramped down over a period of about 8 minutes to an exhaust temperature of about 45° C.

### Example 7

Coated sustained-release tablets of pramipexole dihydrochloride were prepared having the composition shown in Table 6.

TABLE 6
Composition of coated tablets of Example 7

| Ingredient | Amount (mg) |
|---|---|
| pramipexole dihydrochloride monohydrate | 0.375 |
| HPMC type 2208, 4000 mPa s | 140.0 |
| pregelatinized starch | 206.5 |
| colloidal silicon dioxide | 1.4 |
| magnesium stearate | 1.75 |
| total core | 350 |
| ethylcellulose-based coating material (Surelease ®) | 8.4 |
| HPMC-based coating material (Opadry ®) | 2.1 |
| total coating | 10.5 |

Tablet cores were prepared exactly as in Example 5, using pregelatinized starch having a tensile strength of at least about 0.175 kN cm⁻². A coating solution was prepared as follows. Opadry® HPMC-based material in an amount of 4.801 g was added to 103.041 g water and mixed for 45 minutes to provide an HPMC mixture. Next, 76.819 g Surelease® ethylcellulose-based material was added to the HPMC mixture and mixed for an additional 30 minutes to provide a coating solution.

Coating to a 3% weight gain and curing of the coated tablets were performed exactly as in Example 6.

### Example 8

Coated sustained-release tablets of pramipexole dihydrochloride were prepared having the composition shown in Table 7.

TABLE 7
Composition of coated tablets of Example 8

| Ingredient | Amount (mg) |
|---|---|
| pramipexole dihydrochloride monohydrate | 0.375 |
| HPMC type 2208, 4000 mPa s | 140.0 |
| pregelatinized starch | 206.5 |
| colloidal silicon dioxide | 1.4 |
| magnesium stearate | 1.75 |
| total core | 350 |
| ethylcellulose-based coating material (Surelease ®) | 13.13 |
| HPMC-based coating material (Opadry ®) | 4.38 |
| total coating | 17.5 |

Tablet cores were prepared exactly as in Example 5, using pregelatinized starch having a tensile strength of at least about 0.175 kN cm⁻². A coating solution was prepared as follows. Opadry® HPMC-based material in an amount of 10.003 g was added to 177.737 g water and mixed for 45 minutes to provide an HPMC mixture. Next, 120.03 g Surelease® ethylcellulose-based material was added to the HPMC mixture and mixed for an additional 30 minutes to provide a coating solution.

Coating to a 3% weight gain and curing of the coated tablets were performed exactly as in Example 6. After this first curing step, coating was repeated to provide a total tablet weight gain of about 5%, followed by curing for about 15 minutes at a bed temperature of at least about 70° C. After curing, temperature was ramped down over a period of about 8 minutes to an exhaust temperature of about 45° C.

### Example 9

Coated sustained-release tablets of pramipexole dihydrochloride were prepared having the composition shown in Table 8.

TABLE 8
Composition of coated tablets of Example 9

| Ingredient | Amount (mg) |
|---|---|
| pramipexole dihydrochloride monohydrate | 0.375 |
| HPMC type 2208, 4000 mPa s | 140.0 |
| pregelatinized starch | 206.5 |
| colloidal silicon dioxide | 1.4 |
| magnesium stearate | 1.75 |
| total core | 350 |
| ethylcellulose-based coating material (Surelease ®) | 14.0 |
| HPMC-based coating material (Opadry ®) | 3.5 |
| total coating | 17.5 |

Tablet cores were prepared exactly as in Example 5, using pregelatinized starch having a tensile strength of at least about 0.175 kN cm⁻². A coating solution was prepared as follows. Opadry® HPMC-based material in an amount of 8.002 g was added to 171.735 g water and mixed for 45 minutes to provide an HPMC mixture. Next, 128.032 g Surelease® ethylcellulose-based material was added to the HPMC mixture and mixed for an additional 30 minutes to provide a coating solution.

Coating to a 5% total weight gain and curing of the coated tablets were performed exactly as in Example 8.

### Example 10

Dissolution profiles of the 0.375 mg pramipexole dihydrochloride tablets of each of Examples 5, 6 and 9 were evaluated in a standard in vitro USP dissolution assay under the following conditions. USP apparatus 1 was used to stir a dissolution medium (900 ml of 0.05M phosphate buffer at a pH of 6.8) at a spindle rotation speed of 100 rpm and a temperature of 37° C.

Data are shown in FIG. 4. The uncoated tablet of Example 5 and the tablet of Example 6 having a 3% coating comprising 25% pore-former exhibited very similar overall dissolution profiles. On close inspection, however, it will be noticed that the uncoated tablet of Example 5 showed faster initial dissolution, such that at 1 hour and 2 hour sampling times the percent dissolved was greater, than in the case of the coated tablet of Example 6. For example, at 1 hour, the coated tablet of Example 6 showed only 11% dissolution, while the uncoated tablet of Example 5 showed 15% dissolution. Similarly, at 2 hours, the coated tablet of Example 6 showed no more than 20% dissolution, while the uncoated tablet of Example 5 showed 24% dissolution.

Dissolution of the tablet of Example 9 having a 5% coating comprising 20% pore-former exhibited a dissolution profile much slower than either the tablet of Example 5 or the tablet of Example 6.

### Example 11

(S,S)-reboxetine succinate sustained-release tablets were prepared having the compositions shown in Table 9. It will be noted that each tablet comprises 5.5 mg (S,S)-reboxetine succinate, equivalent to 4 mg (S,S)-reboxetine base.

TABLE 9
Composition of (S,S)-reboxetine succinate tablets of Example 11

| Ingredient | Amount (mg) | | |
|---|---|---|---|
| (S,S)-reboxetine succinate | 5.5 | 5.5 | 5.5 |
| HPMC type 2208, 4000 mPa s | 40.0 | 80.0 | 160.0 |
| pregelatinized starch | 53.5 | 112.5 | 230.5 |
| colloidal silicon dioxide | 0.5 | 1.0 | 2.0 |
| magnesium stearate | 0.5 | 1.0 | 2.0 |
| total | 100.0 | 200.0 | 400.0 |

The tablets were prepared by the procedure described in Example 3, using pregelatinized starch having a tensile strength of at least about 0.175 kN cm⁻².

### Example 12

Dissolution profiles of the 4 mg (S,S)-reboxetine tablets of Example 11 were evaluated in a standard in vitro USP dissolution assay under the following conditions. USP apparatus 2 was used to stir a dissolution medium (1 liter of 0.05M phosphate buffer at a pH of 6.8) at a paddle rotation speed of 50 rpm and a temperature of 37° C. The medium was then filtered and samples were analyzed via UV detection.

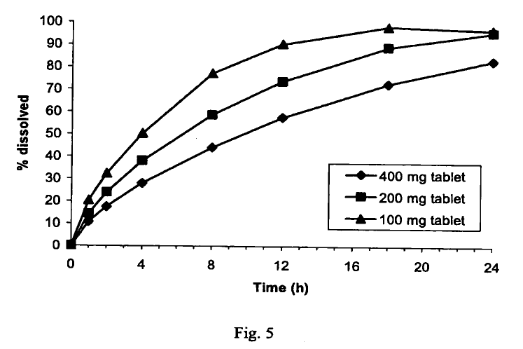

Data are shown in FIG. 5. The tablet having 100 mg total weight exhibited the fastest dissolution, and the tablet having 400 mg total weight the slowest. The tablet having 200 mg total weight was intermediate in dissolution rate.

1-37. (canceled)

38. A pharmaceutical composition in a form of an orally deliverable tablet comprising reboxetine, or a pharmaceutically acceptable salt thereof, dispersed in a matrix comprising a hydrophilic polymer and a starch, wherein the starch has a tensile strength of at least 0.15 kN cm⁻² at a solid fraction of 0.75 to 0.85.

39. The composition of claim 38 wherein the starch has a tensile strength of at least 0.175 kN cm⁻² at a solid fraction of 0.75 to 0.85.

40. The composition of claim 38 wherein the starch has a tensile strength of at least 0.2 kN cm⁻² at a solid fraction of 0.75 to 0.85.

41. The composition of claim 38 wherein the starch is a pregelatinized starch.

42. The composition of claim 38 wherein the starch is present in an amount of about 25% to about 75% by weight of the tablet.

43. The composition of claim 38 wherein the starch is present in an amount of about 40% to about 70% by weight of the tablet.

44. The composition of claim 38 wherein the starch is present in an amount of about 45% to about 65% by weight of the tablet.

45. The composition of claim 38 wherein the hydrophilic polymer is selected from the group consisting of methylcellulose, hydroxypropylmethylcellulose, carmellose sodium and carbomer.

46. The composition of claim 38 wherein the hydrophilic polymer is hydroxypropylmethylcellulose.

47. The composition of claim 38 wherein the hydrophilic polymer is present in an amount of about 20% to about 70% by weight of the tablet.

48. The composition of claim 38 wherein the hydrophilic polymer is present in an amount of about 30% to about 60% by weight of the tablet.

49. The composition of claim 38 wherein the hydrophilic polymer is present in an amount of about 35% to about 50% by weight of the tablet.

50. The composition of claim 38 wherein the reboxetine, or a pharmaceutically acceptable salt thereof, is (S,S)-reboxetine, or a pharmaceutically acceptable salt thereof.

51. The composition of claim 50 wherein the (S,S)-reboxetine, or a pharmaceutically acceptable salt thereof, is (S,S)-reboxetine succinate.

52. The composition of claim 38 that comprises 0.2 to 15 mg reboxetine per tablet.

53. The composition of claim 38 that comprises 1 to 12 mg reboxetine per tablet.

54. The composition of claim 38, further comprising a coating on the tablet.

55. The composition of claim 54 wherein said coating is a release-controlling layer.

56. The composition of claim 55 wherein said release-controlling layer constitutes 1% to 15% by weight of the tablet.

57. The composition of claim 54 wherein said coating is a nonfunctional coating.

58. A pharmaceutical composition in a form of an orally deliverable tablet, comprising (S,S)-reboxetine succinate dispersed in a matrix comprising (a) HPMC in an amount of 35% to 50% by weight of the tablet and (b) a pregelatinized starch having a tensile strength of at least 0.15 kN cm⁻² at a solid fraction of 0.8, in an amount of 45% to 65% by weight of the tablet.

59. A method of treatment of a subject having a central nervous system condition or disorder for which reboxetine, or a pharmaceutically acceptable salt thereof, is indicated, wherein:
the method comprises orally administering to the subject the pharmaceutical composition of claim 38.

60. The method of claim 59 wherein the reboxetine, or a pharmaceutically acceptable salt thereof, is (S,S)-reboxetine succinate.

61. A process for preparing a pharmaceutical composition according to claim 38 in a form of an orally deliverable, sustained-release tablet, the process comprising selecting by a suitable test a starch having a tensile strength of at least 0.15 kN cm⁻² at a solid fraction of 0.75 to 0.85; admixing with the selected starch a hydrophilic polymer and reboxetine, or a pharmaceutically acceptable salt thereof, to provide a mixture wherein the reboxetine, or a pharmaceutically acceptable salt thereof, is dispersed in a matrix comprising the polymer and the starch; and compressing the mixture to form said tablet.

62. The composition of claim 39 wherein the reboxetine, or a pharmaceutically acceptable salt thereof, is (S,S)-reboxetine, or a pharmaceutically acceptable salt thereof.

63. The composition of claim 40 wherein the reboxetine, or a pharmaceutically acceptable salt thereof, is (S,S)-reboxetine, or a pharmaceutically acceptable salt thereof.

64. The composition of claim 41 wherein the reboxetine, or a pharmaceutically acceptable salt thereof, is (S,S)-reboxetine, or a pharmaceutically acceptable salt thereof.

65. The composition of claim 42 wherein the reboxetine, or a pharmaceutically acceptable salt thereof, is (S,S)-reboxetine, or a pharmaceutically acceptable salt thereof.

66. The composition of claim 43 wherein the reboxetine, or a pharmaceutically acceptable salt thereof, is (S,S)-reboxetine, or a pharmaceutically acceptable salt thereof.

67. The composition of claim 44 wherein the reboxetine, or a pharmaceutically acceptable salt thereof, is (S,S)-reboxetine, or a pharmaceutically acceptable salt thereof.

68. The composition of claim 45 wherein the reboxetine, or a pharmaceutically acceptable salt thereof, is (S,S)-reboxetine, or a pharmaceutically acceptable salt thereof.

69. The composition of claim 46 wherein the reboxetine, or a pharmaceutically acceptable salt thereof, is (S,S)-reboxetine, or a pharmaceutically acceptable salt thereof.

70. The composition of claim 47 wherein the reboxetine, or a pharmaceutically acceptable salt thereof, is (S,S)-reboxetine, or a pharmaceutically acceptable salt thereof.

71. The composition of claim 48 wherein the reboxetine, or a pharmaceutically acceptable salt thereof, is (S,S)-reboxetine, or a pharmaceutically acceptable salt thereof.

72. The composition of claim 49 wherein the reboxetine, or a pharmaceutically acceptable salt thereof, is (S,S)-reboxetine, or a pharmaceutically acceptable salt thereof.
